(19) United States
(12) Patent Application Publication
(10) Pub. No.: US 2005/0136112 A1
(43) Pub. Date: Jun. 23, 2005

(54) Oral medicament delivery system

(75) Inventors: Gilbert R. Gonzales, New York, NY (US); Kenneth E. Hughes, Pataskala, OH (US); Sofia O. Gonzales, New York, NY (US)

Correspondence Address: WOOD, HERRON & EVANS, LLP, 2700 CAREW TOWER, 441 VINE STREET, CINCINNATI, OH, 45202, US

(73) Assignee: PediaMed Pharmaceuticals, Inc.

(21) Appl. No.: 10/741,873

(22) Filed: Dec. 19, 2003

(51) Int. Cl. A61K 9/24; A61K 9/14

(52) U.S. Cl. 424/473

(57) ABSTRACT

Oral medicament delivery system comprising a pharmaceutical composition comprising a flexible matrix, said matrix formed of a plurality of fibers comprising a collagen-based carrier and a medicament, the composition orally dissolvable to deliver a unit dose of the medicament to a patient. The flexible composition can be dose titrated and co-administered with a second pharmaceutical formulation.

### BACKGROUND OF THE INVENTION

I. Field of the Invention

The present invention relates to an oral medicament delivery system and, in particular, to a fibrous composition for orally delivering a medicament to a patient.

II. Description of the Prior Art

A common method of delivering a medicament to a patient is through the oral cavity. Commonly utilized orally administrable formulations include solid formulations such as tablets, pills, capsules, oral dispersible pills, dragees, troches, lozenges and the like. However, administering medicaments via such solid formulations presents challenges and drawbacks to a portion of the patient populations. Statistically, at least 20 percent of the population finds it difficult to swallow solid oral medicament formulations and, consequently, are averted to ingesting such formulations. Further, about 10 percent of women are completely unable to swallow intact tablets, pills, and capsules, without severing them into smaller pieces.

Patients are generally reluctant and/or averted to swallowing a solid medicament formulation particularly when the act of swallowing is problematic for the individual. Complications such as global hystericus and/or choking due to pharyngeal and esophageal motility problems, generally render it painful for a patient to swallow. In addition, patients with pharyngitis and/or a markedly swollen or an otherwise severely irritated pharynx, such as due to a bacterial infection, often makes it difficult and/or impossible for the patient to swallow. Chronic disorders and other conditions, such as a psychological and/or psychosomatic aversion to the act of swallowing or a fear of choking on pills and tablets, render patients even more reluctant to swallow solidly formulated, non-chewable medicaments. Patients may also be reluctant to swallow solid medicament formulations due to their physical properties, including size, shape, and/or taste, or simply because of personal choice not to swallow and/or ingest the formulation.

Members of the pediatric patient population are reluctant to swallow solid medicament formulations for additional reasons. Particularly, small children generally dislike ingesting pills, tablets, and other “medicines.” In addition, children generally refuse to orally ingest a medication during periods of illness. Further, children are generally more inclined to chew pills and tablets than swallow them, typically experiencing a bitter taste in their mouths, which frequently causes children to either spit out or discard the formulation and otherwise refuse to ingest it. Also, many of the available pills and tablets are scored in halves only and not in smaller portions, such as thirds or quarters, thereby rendering dose titration of those pediatric medications difficult and sometimes impossible. Accordingly, the child must swallow a larger portion of those solid medications to obtain the physician directed and/or recommended dose. Finally, it is even more difficult to co-administer a second solid medicament formulation, which may be necessary to provide a specific benefit such as to enhance the dose of the first medicament, to a child with a physical and/or mental aversion to swallowing a solid, non-chewable medication.

More recently, chewable and dissolvable forms of a medication have been proposed. For example, U.S. Pat. Nos. 4,855,326, 4,997,856, 5,034,421, and 5,096,492, each disclose a floss-type melt-spun pharmaceutical composition having a sugar-based carrier or a sugar-oleagenous combination carrier for delivering a medicament to a patient. However, due to physical properties of the carrier materials, these compositions were found not to be very stable for long periods of times. Particularly, being susceptible to the degradative effects of humidity, such formulations have not been extremely successful and/or extensively commercialized. More specifically, many medicaments were found not to be stable with these carriers, thereby raising the costs associated with that medication.

However, patients, whether a child or adult, under a medication regimen and/or simply in need of the therapeutic benefits of a medicament must be administered, or self-administer, the dosage formulation.

Thus, there is a need to provide a better method for orally administering a medicament to a patient. There is a further need to orally administer medicaments in a manner that addresses patient reluctance and/or aversion to swallowing and/or ingesting a medicament formulation. There is a further need to improve patient ingestion of a medicament in compliance with a medication regimen, and in particular, improved compliance by pediatric patients. There is yet a further need to provide a medicament formulation that provides stability for a wide variety of medicaments.

### SUMMARY OF THE INVENTION

The present invention provides an oral medicament delivery system comprising a fibrous pharmaceutical composition, for orally delivering a medicament to a patient. The delivery system addresses weaknesses and drawbacks associated with previously proposed oral medicament delivery formulations and, in particular, addresses the drawbacks associated with previously proposed, and commonly utilized, solid medicament formulations. Particularly, the delivery system addresses the problems associated with swallowing pills, tablets, and other conventional solid medicament formulations, as described in the background section of the invention. In addition, the delivery system addresses the drawbacks associated with proposed sugar-based floss as carriers for medicaments. Further, the pharmaceutical compositions, as part of the delivery system, do not resemble a pill or tablet but have a fibrous appearance and structure, microscopically, which renders the composition mucous membrane adhesive, flexible and orally dissolvable and, therefore, more acceptable and desirable to adult patients, and non-expectorable by pediatric patients. Furthermore, this dose delivery form can be torn, cut or severed with scissors to produce smaller dosage forms i.e., halves, quarters or other sizes).

The pharmaceutical composition in the oral medication delivery system generally comprises a matrix formed of fibers, wherein some or all of the fibers include a collagen-based carrier and a medicament. The fibrous matrix is capable of partially or completely dissolving in the oral cavity to deliver a dose of the medicament, transmucosally or via the gut, to the patient. The basic unit of the medicament-containing composition is a fiber, strand or filament, as opposed to the micron-size particles and/or granules in conventional pressure-compacted tablets.

The fiber in the composition is collagen-based, i.e., it is formed primarily of collagen protein, and the medicament is incorporated therein or distributed thereon in various embodiments. Thus, the collagen-based fiber serves as a carrier or vehicle for delivering the medicament. Collagen provides many advantages as a carrier. Specifically, collagen has proven success in many bodily applications. Collagen is also capable of binding and carrying charged active pharmaceutical ingredients (API). Collagen is relatively cheap, readily available and its purity and sterility can be controlled. Collagen can withstand aseptic processing techniques at mild temperatures ranging from about 20° C. to about 35° C., which many API's can withstand without decomposition.

In embodiments of the invention, the medicament may be distributed on, or incorporated in, each fiber, or a selected group of fibers, and may be any compound providing a biological and/or therapeutic benefit to the patient. Thus, exemplary medicaments include, without limitation, active pharmaceutical ingredients (API), and non-actives such as vitamins, minerals and the like. The amount of the medicament is selected as desired, and generally depends upon the particular medicament, accepted dosing practices for that medicament, purpose of administration, and targeted patient population. The amount of the medicament may also be dependant upon the amount, by weight, of the carrier fiber and its inherent porosity and absorptive nature. Such dependency allows the composition to be administered in a dose that can be titrated and/or generally monitored, in accordance with the recommended dose.

In one embodiment, the composition, or the individual fibers, further include excipients to provide desirable aesthetic, physical and/or chemical properties to the orally administrable composition. For example, dissolvable excipients including water-soluble substances including basic salts or buffers, which generally dissolve in saliva (mildly acidic) or in water-containing fluids in the oral cavity may be included in the composition. Simple sugars and combinations thereof including mono-saccharides, di-saccharides and poly-saccharides and other sweeteners generally provide a sweet taste, thereby rendering the composition further appealing to children. Taste masking components may also be added to improve taste and/or to overcome offensive bitter aftertastes from chewing and/or ingesting various broken or cut tablets, pills and capsules, which were otherwise intended to be swallowed.

Embodiments of the composition may also include a bio-adhesive or muco-adhesive to adhere the composition to the patient's buccal mucous membrane. Such adhesion allows the composition to be exposed for a period of time, while retained on the oral mucous membrane, to appropriate dissolution conditions, thereby dissolving the composition over time and providing a delayed release effect for delivering the medicament to the patient. In addition, a muco-adhesive is advantageous for medicaments that are more effective when absorbed across the mucosal membrane, thereby bypassing the hepatic first pass effects.

The composition of the medicament delivery system is manufactured using non-spun, non-melted methods, and is fibrous in nature, formed from a flexible matrix of fibers. The matrix may be shaped into any suitable, or desirable, form for oral ingestion. For example, the composition may exist in a coin-like circular shape the size of a dime or quarter. A “fusiform” shape is also contemplated, i.e., an elongate shape having a thick or broad central region and thin terminal regions. Generally, most desired shapes may be prepared by either (1) forming sheets of fibers, stacking multiple sheets one on top of the other to form layers, and then compacting and compressing these layers into a unique shape, or (2) casting solutions of the matrix collagen material, with or without the medicament added, allowing the collagen to form a fibrous gel matrix, then dehydrating, pressing, die-cutting, or otherwise processing the gel. The composition may also be marked to indicate a dosage time and/or schedule, and/or perforated or scored to allow the composition to be easily severed.

The fibrous nature of the composition provides advantages over conventional pills, tablets, capsules, oral dispersal forms (i.e. “melting tablets”) and other solid oral dosage formulations. Particularly, it allows the composition to be easily cut and severed, unlike most conventional tablets, pills and capsules. This benefit provides the ability to titrate and/or monitor the dose administered to the patient. The chewable and dissolvable nature of the composition render it more likely to be ingested by pediatric patients, who may otherwise be physiologically and/or psychologically reluctant to swallow a conventional solid dosage formulation. Further, in accordance with another aspect of the invention, the flexibility of the fibrous matrix allows the composition to be wrapped around other dosage formulations, such pills and tablets, for co-administration of multiple medicaments to a patient. For example, the fibrous matrix contains one or more API's while a pill or tablet or other dosage wrapped therein includes other different API's. Alternatively, the fibrous matrix includes an API for rapid delivery and a solid dosage wrapped therein includes the same or similar API for more delayed release. In another embodiment, compositions having mucoadhesive properties also prevent the pediatric patient, non-compliant patient, and/or obstreperous patient from spitting the composition out of their mouths. In addition, unlike oral dispersible tablets, such as Zydis® formulations and others, the present fibrous composition is not API dose limited.

The present invention also provides methods of forming the fibrous medicament-containing composition and methods for administering it to a patient. The composition may be administered directly, by placing it in the patient's oral cavity, or indirectly by, for example, first suspending or dissolving it in an amount of a liquid, such as water, juice or other patient selected beverage, in a spoon, glass, cup or other vessel of choice. In either method, the composition dissolves and disperses the medicament in the liquid (or saliva) prior to ingestion by the patient. Conventional pills and tablets generally do not dissolve in saliva or a chosen liquid. Accordingly, the oral medicament delivery system of the present invention encourages ingestion of a medicament, in compliance with a medication regimen. Particularly, the composition in liquid form is easily swallowed and is generally not thereafter retained and later expelled from the mouth. In this manner, the present invention also improves compliance in psychiatric and/or other non-compliant patients.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is one exemplary form of the pharmaceutical composition in the present invention;

FIG. 2 is a second exemplary form of the pharmaceutical composition in the present invention;

FIGS. 3A-3F illustrate a first exemplary method of making the medicament-containing, collagen-based composition of the present invention;

FIG. 4 is a perspective, exploded view of the product made by the exemplary method illustrated in FIGS. 3A-3F;

FIG. 5 is a perspective view the compressed product made by the exemplary method illustrated in FIGS. 3A-3F;

FIG. 6 illustrates a second exemplary method of making the medicament-containing, collagen-based composition of the present invention; and

FIG. 7 is a partial cut-away view of the product made by the exemplary method illustrated in FIG. 6.

### DETAILED DESCRIPTION OF EXEMPLARY EMBODIMENTS

The present invention will be further appreciated in view of the following definitions:

The term “system” with respect to delivery of a medicament, is generally intended to refer to one or more compositions delivered to the patient. Thus, this term contemplates delivery of a single pharmaceutical composition, such as the fibrous composition alone, or a combination of pharmaceutical formulations, such as the fibrous composition with one or more other formulations, administered simultaneously or sequentially one after another.

The term “matrix”, as used herein, is intended to generally refer to a weave or web of fibers, strands, threads or filament-like structures. For example, the fibers may be inter-woven, cross-woven, or arranged in any other orientation relative one another to form a matrix, as appreciated by one of ordinary skill in the art.

The term “collagen-based”, as used herein with reference to the composition of the carrier, is intended to generally refer to a material that is primarily comprised of a form of collagen. Thus, this term contemplates 100% collagen carrier, and also carriers having at least 50% by weight of a collagen material. This term also contemplates all forms of collagen, natural or synthetic, including homo-polymers, cross-linked polymers (tropocollagen), copolymers and strands of collagen, as well as collagen-type proteins. Accordingly, this term also encompasses chemically modified collagen, formed by known synthetic methods such as by replacing one or more of the amino acids in the collagen protein backbone, or modifying the side chains of the polymer(s).

The term “carrier”, as used herein, is intended to generally refer to a vehicle or substrate for delivering the medicament to the patient.

The term “medicament”, as used herein, is intended to generally refer to any biological substance having a physiological and/or therapeutic effect on a patient. For example, the term “medicament” encompasses all active pharmaceutical ingredients (API), non-active ingredients including vitamins, minerals, dietary components, and the like. This term also encompasses compounds, which are administered for their therapeutic and/or prophylactic effects.

The terms “orally dissolvable” and “orally dispersible”, as used herein with reference to the composition, is intended to generally refer to the breakdown, up to and including complete dissolution, of the composition. Thus, this term encompasses compositions, which partially or fully breakdown or dissolve in a particular medium, such as saliva, or aqueous liquids and foods, present in the oral cavity. As such, this term encompasses all compositions, which will generally dissolve in the oral environment stimulated with liquid or other substance to generate the secretion of saliva.

The present invention provides an oral medicament delivery system comprising a fibrous pharmaceutical composition, and also provides methods for orally delivering a medication to a patient. The pharmaceutical composition is generally a fibrous matrix formed of a plurality of fibers. The composition is sufficiently dissolvable in the oral cavity to deliver a unit dose of the medicament to the oral mucosal membrane, or the gut via ingestion, of the patient.

Each fiber may generally serve as a carrier to deliver the one or more medicaments to the patient's oral cavity. The carrier is generally formed of collagen or a collagen-based material. Collagen is a naturally occurring fibrous protein formed of fibers having a high tensile strength, and whose solubility in aqueous and organic media, such as alcohol and ether, varies depending upon the particular structure and form of the collagen. Collagen provides many advantages as a carrier. Particularly, collagen has been successful in physiological applications and uses, including use as implants and hemostatic agents in the medical industry, as topical agents in the cosmetic industry, and as ingestible products in the food industry. Collagen may possibly be charged thereby enhancing its binding and/or carrying capability for charged medicaments, and particularly charged active pharmaceutical ingredients (API). Collagen is relatively cheap and readily available in a multitude of sources and raw materials and, therefore, provides a low cost base material, relative to many other formulation materials. In addition, the purity of the collagen can be controlled.

Further, the processing of collagen lends itself to pharmaceutical formulation by withstanding aseptic techniques and by being readily processed at mild temperatures ranging from about 20° C. to about 35° C. Such ambient temperature allows for the inclusion of many API's without the risk of degradation and/or chemical decomposition, which may otherwise result from exposure to higher temperatures. Also, collagen is a suitable extrusion candidate for the medicament carrier material. Once in solution, collagen can easily be strained of its solvent liquid and extruded to form rapidly drying and solidifying fibrous strands or threads. Alternatively, collagen in solution may be induced to form a fibrous gel matrix by a process called reconstitution. These gels may be further processed to yield non-woven fibrous matrices having a highly porous structure.

Moreover, the physical properties of collagen are desirable. Specifically, many forms of collagen are hydroscopic and, therefore, soluble in saliva and most other fluids, and foods, typically present in the oral cavity. The mechanical and chemical properties of collagen matrices can be varied during processing to control their handling and dissolution properties. This can be accomplished by such means as using various salts, acids or enzymes during the original collagen extraction process, by modifying the extrusion of gel reconstitution process, or by using cross-linking agents or other treatment methods during production of the collagen matrix. Thus, collagen or a collagen-based material as the carrier provides many properties particularly useful for an orally administrable medicament formulation.

The amount of collagen in each strand of fiber may vary depending on the desired properties of the administrable composition. For example, being hygroscopic, highly porous, and generally soluble in aqueous-based liquids, including saliva, large amounts of the collagen-based carrier are not necessary. The amount of collagen may also be based upon the amount of the medicament(s) carried within or distributed on the fiber, as is discussed herein.

The medicament associated with the fiber, and the composition as a whole, may be selected as desired. Suitable medicaments include active pharmaceutical ingredients (API). For example, the medicament may be any of the following API's, many of which are well-known drugs:

Analgesic anti-inflammatory agents, such as acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like;

Drugs having an action on the central nervous system, for example sedative agents, hypnotic agents, anti-anxiolytic agents, analgesic and anesthetic agents, such as chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivacaine, bupivacaine, etidocaine, prilocalne, benzocaine, fentanyl, nicotine, morphine, codeine, hydrocodone, hydromorphone, diacetylmorphine, methadone, sufentanyl, meperidine, levodromoran, and the like;

Antihistaminic or anti-allergenic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, chlorpheniramine, phenylpropanolamine, phenylephrine, atropine, hyoscyamine, cyproheptidine, and the like;

Anti-inflammatory agents including steroids, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, androgenic steroids, such as, testosterone, methyltestosterone, fluoxymesterone, estrogens such as, conjugated estrogens, esterified estrogens, estropipate, 17-beta estradiol, 17-beta estradiol valerate, equilin, mestranol, estrone, estriol, 17-beta ethinyl estradiol, and diethylstilbestrol; progestational agents such as progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17-alpha hydroxyprogesterone, dydrogesterone, dimethisterone, ethinylestrenol, norgestrel, demegestone, promegestone, megestrol acetate, and the like;

Respiratory agents such as theophilline and beta₂-adrenergic agonists such as albuterol, terbutaline, metaproterenol, ritodrine, carbuterol, fenoterol, quinterenol, rimiterol, solmefamol, soterenol, tetroquinol, caffeine, caffeine citrate, and the like;

Sympathomimetic agents, such as dopamine, norepinephrine, phenylpropanolamine, phenylephrine, pseudoephedrine, amphetamine, propylhexedrine, arecoline, and the like;

Local anesthetics agents, such as benzocaine, prilocalne, bupivocaine, procaine, dibucaine, lidocaine, and the like;

Antimicrobial agents including antibacterial agents, antifungal agents, antimycotic agents and antiviral agents; tetracyclines such as, oxytetracycline; penicillins such as ampicillin; cephalosporins such as cefalotin; aminoglycosides such as kanamycin; macrolides such as erythromycin, chloramphenicol, iodides, nitrofrantoin, nystatin, amphotericin, fradiomycin, sulfonamides, pyrrolnitrin, clotrimazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like;

Antihypertensive agents such as clonidine, alpha-methyldopa, reserpine, syrosingopine, rescinnamine, cinnarizine, hydrazine, prazosin, ACE inhibitors, propanolol, pindolol, labetalol, clonidine, captopril, enalapril, lisonopril, and the like;

Antihypertensive diuretics such as chlorothiazide, hydrochlorothrazide, bendoflumethazide, trichlormethiazide, furosemide, tripamide, methylclothiazide, penfluzide, hydrothiazide, spironolactone, metolazone, and the like;

Cardiotonic agents such as digitalis, ubidecarenone, dopamine, and the like;

Coronary vasodilators such as organic nitrates including, without limitation, nitroglycerine, isosorbitol dinitrate, erythritol tetranitrate, pentaerythritol tetranitrate, dipyridamole, dilazep, trapidil, trimetazidine, and the like;

Vasoconstrictors, such as dihydroergotamine, dihydroergotoxine, and the like;

Beta-blockers or antiarrhythmic agents, such as timolol pindolol, propranolol, and the like;

Calcium antagonists and other circulatory organ agents including, without limitation, aptopril, diltiazem, nifedipine, nicardipine, verapamil, bencyclane, ifenprodil tartarate, molsidomine, clonidine, prazosin, and the like;

Anti-convulsive agents such as nitrazepam, meprobamate, phenobarbitol, carbomazepine, valproic acid, oxazepine, phenyloin, and the like;

Agents for dizziness and nausea such as isoprenaline, betahistine, scopolamine, and the like;

Tranquilizing agents such as reserprine and chlorpromazine; and antianxiety benzodiazepines, such as alprazolam, chlordiazepoxide, clorazeptate, halazepam, oxazepam, prazepam, clonazepam, flurazepam, triazolam, lorazepam, diazepam, and the like;

Antipsychotic agents such as butyrophenones and phenothiazines including, without limitation, thiopropazate, chlorpromazine, triflupromazine, mesoridazine, piperracetazine, thioridazine, acetophenazine, fluphenazine, perphenazine, trifluoperazine, and other major tranqulizers such as chlorprathixene, thiothixene, haloperidol, bromperidol, loxapine, and molindone, as well as those agents used at lower doses in the treatment of nausea, vomiting, and the like;

Muscle relaxants, such as tolperisone, baclofen, dantrolene sodium, cyclobenzaprine, and the like;

Drugs for Parkinson's disease, spasticity and acute muscle spasms, such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, and the like;

Respiratory agents and cough suppressants such as codeine, ephedrine, isoproterenol, dextromethorphan, orciprenaline, ipratropium bromide, cromglycic acid, and the like;

Non-steroidal hormones or antihormones such as corticotropin, oxytocin, vasopressin, salivary hormone, thyroid hormone, adrenal hormone, kallikrein, insulin, oxendolone, and the like;

Antitumor agents such as 5-fluorouracil and derivatives thereof, krestin, picibanil, ancitabine, cytarabine, and the like;

Enzymes such as lysozyme, urokinaze, and the like;

Herb medicines or crude extracts such as glycyrrhiza, aloe, Sikon (Lithospermi Radix), and the like;

Miotic agents such as pilocarpine, and the like;

Cholinergic agonists such as choline, acetylcholine, methacholine, carbachol, bethanechol, pilocarpine, muscarine, arecoline, and the like;

Antimuscarinic or muscarinic cholinergic blocking agents such as atropine, scopolamine, homatropine, methscopolamine, homatropine methylbromide, methantheline, cyclopentolate, tropicamide, propantheline, anisotropine, dicyclomine, eucatropine, and the like;

Mydriatic agents such as atropine, cyclopentolate, homatropine, scopolamine, tropicamide, eucatropine, hydroxyamphetamine, and the like;

Psychic energizers such as 3-(2-aminopropy)indole, 3-(2-aminobutyl)indole, and the like;

Humoral agents such as the prostaglandins, natural and synthetic, for example, PGE₁, PGE₂alpha, and PGF₂alpha, and the PGE₁ analog misoprostol.

Antispasmodic agents such as atropine, methantheline, papaverine, cinnamedrine, methylscopolamine, and the like;

Antidepressive agents such as isocarboxazid, pheneizine, tranylcypromine, imipramine, amitriptyline, trimipramine, doxepin, desipramine, nortriptyline, protriptyline, amoxapine, maprotiline, trazodone, and the like;

Anti-diabetic agents such as insulin, and anticancer drugs such as, tamoxifen, methotrexate, and the like;

Anorectic drugs such as dextroamphetamine, methamphetamine, phenylpropanolamine, fenfluramine, diethylpropion, mazindol, phentermine, and the like;

Anti-allergenic agents such as antazoline, methapyrilene, chlorpheniramine, pyrilamine, pheniramine, and the like;

Decongestants such as phenylephrine, ephedrine, naphazoline, tetrahydrozoline, and the like;

Antipyretic agents such as aspirin, salicylamide, and the like;

Antimigrane agents such as dihydroergotamine, pizotyline, triptans, and the like;

Anti-malarial agents such as the 4-aminoquinolines, alphaminoquinolines, chloroquine, pyrimethamine, and the like;

Anti-ulcerative agents such as misoprostol, omeprazole, enprostil, and the like;

Peptides such as growth releasing factor, and the like;

Anti-estrogen or anti-hormone agents, such as tamoxifen or human chorionic gonadotropin, and the like; and

Antiulcer agents such as allantoin, aldioxa, alcloxa, methylscopolamine methylsuflate, and the like.

The exemplary medicaments and drugs listed above may be used individually or in combination as required. Moreover, the drugs may be used either in their free-base form or, if capable of forming salts, in the form of a salt with a suitable counter ion, such as a suitable acid or base. Suitable acidic counter ions include, without limitation, organic acids such as methane sulfonic acid, toluene sulfonic acid, lactic acid, tartaric acid, fumaric acid, maleic acid, succinic acid, acetic acid and the like, and inorganic acids, such as hydrochloric acid, hydrobromic acid, hydrofluoric acid, phosphoric acid, sulfuric acid and the like. Suitable basic counter ions include, without limitation, organic bases such alkyl amines including triethylamine, and the like, and inorganic bases, such as sodium hydroxide, potassium hydroxide, lithium hydroxide, calcium hydroxide, ammonia and the like. If the medicament has a carboxylic acid functional group, then derivatives of the acid, such as an ester, an anhydride or an amide, may be employed. The esters may include alkyl esters, aryl esters, aralkyl esters, and the like, and may also have functional groups capable of themselves forming salts.

The medicament may also be a nutritional ingredient such as a vitamin, mineral, and the like. The term “vitamin”, as used herein, includes, without limitation, thiamin, riboflavin, nicotinic acid, pantothenic acid, pyridoxine, biotin, folic acid, vitamin B₆, vitamin B₁₂, lipoic acid, ascorbic acid, vitamin A, vitamin D, vitamin E, vitamin K and derivatives thereof, calciferols, mecobalamin, and the like. Also included within the term “vitamin” are the coenzymes thereof, as coenzymes are generally beneficial agents for the body. Coenzymes include thiamine pyrophosphates (TPP), flavin mononucleotides (FMM), flavin adenine dinucleotides (FAD), Nicotinamide adenine dinucleotides (AND), Nicotinamide adenine dinucleotide phosphate (NADP), Coenzyme-A (CoA) pyridoxal phosphate, biocytin, tetrahydrofolic acid, coenzyme B₁₂, lipoyllysine, 1,1-cis-retinal, and 1,2,5-dihydroxycholecalciferol. The term “vitamin” also includes choline, carnitine, and alpha, beta, and gamma carotenes. Thus, a vitamin may include, for example, substances that may or may not be required in the diet. Salts of vitamins are also suitable.

The term “mineral”, as used herein, refers to inorganic substances, such as metal compounds and the like, generally required in the diet. Thus, suitable minerals include, without limitation, calcium, iron, zinc, selenium, copper, iodine, magnesium, phosphorus, chromium and the like, their salts, chelates, and other compositional forms and combinations thereof.

Other nutritional ingredients, commonly referred to as “dietary supplements”, include substances which have an appreciable nutritional effect when administered in small amounts. Suitable dietary supplements include, without limitation, ingredients such as bee pollen, bran, wheat germ, kelp, cod liver oil, ginseng, and fish oils, amino acids, proteins and mixtures thereof. It should be appreciated that dietary supplements may also incorporate vitamins and minerals.

The medicament may be incorporated in each fiber. For example, each fiber may be formed, such as by extrusion or other methods, with the medicament. Alternatively, the medicament may be distributed on each fiber or over a collection of fibers, in the composition. For example, individual fibers or a grouping of fibers may be coated with the medicament.

The amount of the medicament included in the formulation will generally depend upon the particular medicament, its intended use, and patient profile. The medicament is dosed in accordance with accepted pharmacy and FDA practices and government regulations. For example, medicament compositions targeted for administration to children will include the medicament in smaller amounts such as from 1 mg to about 25 mg, which encompass therapeutically effective doses for a majority of pediatric medicaments, and generally sufficient dosages for many adult medications. Effective dosages are generally that amount or quantity of a drug or pharmaceutically active substance, which is sufficient to elicit the required or desired therapeutic response (biological response) when administered to a patient. In one embodiment, the composition includes the medicament(s) in dosage amounts of up to about 1000 mg. In another embodiment, the composition includes the medicament(s) in dosage amounts ranging from about 25 mg to about 100 mg. In yet another embodiment, the composition includes the medicament(s) in dosage amounts of up to about 25 mg. Larger dosages will generally increase the size of the fibrous matrix. But this is not a disadvantage, as the composition may be dissolved, broken down and otherwise delivered in a suitable liquid or aqueous medium of the patient's choice.

The amount of the medicament may also be dependent on the amount of the collagen-based carrier, and vice versa, in the composition. For example, depending upon physical properties and degree of dispersion and solubility in the oral environment desired, the quantities and weight ratios of the carrier to the medicament are varied. In one embodiment of the invention, the carrier to medicament weight ratio in the composition, or in each fiber, is in the range from about 50:1 to about 1:50. In another embodiment, the carrier to medicament weight ratio is in the range from about 10:1 to about 1:10. In yet another embodiment, the carrier to medicament weight ratio is about 1:1.

With reference to a vitamin or mineral, an effective amount is generally at least about 10% of the United States recommended Daily Allowance (“RDA”) of the particular ingredient for a patient. For example, an effective amount of vitamin C would include an amount of vitamin C sufficient to provide 10% or more of the RDA. Typically, where the formulation includes a vitamin or mineral, it will incorporate higher amounts, such as about 100% or more of the applicable RDA.

The composition as a whole, each fiber, or a selected collection of fibers, may further include other desirable excipients. Excipients that dissolve in the oral environment are useful. Suitable excipients include, without limitation, carbohydrates, mono-saccharides, di-saccharides, poly-saccharides of simple sugars, sugar derivatives, and the like. Examples of suitable sugars and other excipients include, without limitation, high caloric sugars such as sucrose, lactose, glucose, d-glucose, l-glucose, maltose, dextrose, fructose, fructosan, gentiobiose, cellobiose, panose, malto-triose, malto-tetrose, arabinose, mannose, d-mannose, galactose, d-galactose, d-glyceraldehyde, amylose, allose, altose, talose, gulose, idose, ribose, erythrose, threose, lyxose, xylose, d-xylose, rhamnose, invert sugar, corn sugar, inositol, glycerol, glycogen, pectin, agar, sorbitol, mannitol and combinations thereof; low caloric sugars, such as sucralose, polyols, tagarose, trehalose, xylitol, dextrans, dextrins, dextrates, polysorbates, maltodextrin, xylitol, amylase, amylopectin, ribose, β-maltose, fucose, sialic acid (neuraminic acid), N-acetylgalactosamine, N-acetylglucosamine, sedoheptulose, ribulose, xylulose and combinations thereof; non-sugar sweeteners, such as acesulfane potassium, aspartame, neotame, saccharin, stevioside and combinations thereof; non-sweeteners, such as alitame, cyclamate, dihydrchalcones (DHCs), glycyrrhizin, thaumatin, gelatin, glycerin, triacetin, trehalose, alginates, gellan gum, cellulose, microcrystalline cellulose, xanthan gum, cellulose acetate phthalate, hydropropylcellulose, hydropropylmethylcellulose, ethylcellulose, methylcellulose, L-HPC (low-substituted hydroxypropyl cellulose), carrageenan, croscarmellose, povidone, crospovidone, starch, sodium starch glycolate, glucan, Adjumer® (polyidi[carboxylatophenoxyl[phosphazene), Pleuran (glycan), Pluronic L 121 (Poloxamer 401), glyceraldehydes, dihydroxyacetone and combinations thereof; and combination carriers/floss/menstruum, such as without being limited to, directly compressed dried honey (Hony-TAB®), lactose and aspartame, lactose and cellulose, microcrystalline cellulose and carrageenan, microcrystalline cellulose and guar gum, microcrystalline cellulose and sodium carboxymethylcellulose, microcrystalline cellulose and lactose, and a sugar and starch combination.

Desirable adjuvants including, without limitation, binders, non-effervescent disintegrants, coloring agents, flavors, taste enhancers, taste maskers, oral dispersing agents, stabilizers, preservatives, diluents, filler, compaction agents, bioadhesives, effervescent disintegration agents, and the like, may also be included.

Examples of binders include acacia, tragacanth, gelatin, starch, cellulose materials such as methyl cellulose and sodium carboxy methyl cellulose, alginic acids and salts thereof, magnesium aluminum silicate, polyethylene glycol, guar gum, polysaccharide acids, bentonites, sugars, invert sugars and the like. Binders may generally be used in an amount up to about 60% by weight and advantageously from about 10% to about 40% by weight of the total composition.

Furthermore, one or more disintegrants or dispersion enhancers can be used to enhance the breakability of the composition in an aqueous environment, such as the oral cavity. Disintegrants include starches as corn starch, potato starch and modified starches thereof, sweeteners, clays such as bentonite, micro-crystalline cellulose (even as high HLB emulsifier surfactants), purified wood cellulose, alginates, polyvinylpyrrolidones, gums such as agar, guar, partially hydrolyzed guar gum, locust bean, karaya, kaolin, pecitin, sodium starch glycolate, isoamorphous silicate, and tragacanth. Disintegrants may generally comprise up to about 20% by weight and advantageously between about 2% and about 10% by weight of the final composition.

Coloring agents may include titanium dioxide, and dyes suitable for food such as those known as F.D. & C. dyes and natural coloring agents such as grape skin extract, beet red powder, beta-carotene, annato, carmine, turmeric, paprika, etc. The amount of the coloring agent(s) used may range from about 0.1% to about 3.5% by weight of the final administrable composition.

Flavors incorporated in the composition may be chosen from synthetic flavor oils and flavoring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple pear, peach, strawberry, raspberry, cherry, plum, pineapple, apricot and so forth. Flavors, which have been found to be particularly useful, include commercially available orange, grape, cherry and bubble gum flavors and mixtures thereof. The amount of flavoring may depend on a number of factors, including the organoleptic effect desired. Flavors may be present in an amount ranging from about 0.5% to about 3.0% by weight of the composition. Commonly accepted flavors include grape and cherry flavors, and citrus flavors such as orange. It is also appreciated that inclusion of flavoring agents can also influence the final flavor of the vehicle, furthering compliance with ingestion of the medicament.

In accordance with another aspect of the invention, a bioadhesive, such as a bioadhesive polymer, generally increases the contact time between the composition and the oral mucosa, particularly where the composition is administered directly into the oral cavity and the dissolving medium is saliva with the mucoadhesive properties, the inventive compositions are particularly useful for difficult patients. Specifically, pediatric patients, noncompliant patients and/or obstieperous patients are hindered or prevented from spitting the composition out of their mouth. This provides more accurate dosing and compliance with a desired dosing regimen. Non-limiting examples of known bioadhesives, or mucoadhesives, include carbopol (various grades), sodium carboxy methylcellulose, methylcellulose, polycarbophil (Noveon AA-1), hydroxypropyl methylcellulose, hydroxypropyl cellulose, sodium alginate, and sodium hyaluronate.

In another aspect, one or more effervescent disintegration agents might be used. Effervescent disintegration agents generally include at least one acid, such as citric acid, tartaric acid, malic aicd, fumaric acid, adipic acid, succinic acid, acid anhydrides, acid salts and mixtures thereof, and at least one base or a source of carbonate, such as from alkali-metal carbonate salts, bicarbonate salts, and mixtures thereof. The reaction of the acid and base produce gas or cause effervescence in the oral cavity or in the liquid used to dissolve the composition prior to ingestion. With carbonate bases, carbon dioxide gas is produced. The action of such an agent can often aid in masking objectionable tastes of active pharmaceutical ingredients, vitamins, minerals, and other medicaments. Generally, the positive organoleptic sensation achieved by the production of gas in the mouth, combined with the texture, speed and sensation of resulting effervescence also generally masks undesirable flavors in the mouth.

Where effervescent agents are included, they may be included in the composition in various different ways. One method includes incorporating the entire effervescent agent in the fibrous matrix used to form the composition. Another manner of incorporating an effervescent disintegrating agent is to include the entire agent as an additive, which is mixed with fibrous matrix after it is formed. Another method contemplates incorporating one portion of the disintegrating agent in the fibrous matrix and another portion of the disintegrating agent as an additive after formation of the fibrous composition.

The components of the composition, i.e., the collagen-based carrier and the medicament(s) and other desired excipients, where incorporated into the fiber, are processed or converted to fibrous, string-like, threads. Known technology utilized to form the fibrous backbone of the composition is one of creating threads or floss plus the active medicament. For example, the components may be combined into threads or floss in an admixed fashion, wherein the carrier and the medicament, plus other excipient(s) would also need to be added such that any small or large portion of the finally formulated fibrous matrix, or any single portion of a thread or of a fiber, would contain a ratio of the above-mentioned ingredients in a desired proportion to the ratio of all of the ingredients of the composition. Such ratios will be formed as desired for the purpose of dose titration and to secure accurate and reproducible dosing of the medicament to a patient.

Known conventional methods of forming fibers and threads including extrusion, co-extrusion, gel casting, flash-drying and freeze-drying techniques may be employed. Many collagen materials are available commercially, or may be extracted following well-recognized biochemical practices. A suitable method for extraction and reconstitution from a source is disclosed in U.S. Pat. No. 6,197,934, which patent disclosure is incorporated herein by reference in its entirety. Generally, the collagen is acid extracted, salt extracted, or chemically and enzymatically solubilized and extracted in a molecular or small aggregate state, from the raw material source. Such extracts are typically stabilized in a mildly acidic solution and kept refrigerated for longer shelf life.

Reconstitution causes the collagen molecules or aggregates to self-assemble into a fibrous structure, often resembling its original native state. The reconstitution (or self-assembly) process may be induced by titrating the collagen solution to conditions approaching its physiological state (pH 7.0-7.4 and 20-35° C. in an ionically balanced phosphate buffer). Alternatively collagen may be made to precipitate by rapidly increasing the pH above 7.4 and removing water from the solution, thereby increasing the concentration of solids.

Prior to, during, or after reconstitution or precipitation, a desired and compatible medicament, such as an API, is then added to the collagen. Then the collagen may be subjected to a compression process wherein the reconstituted collagen (RC)-medicament mixture is squeezed in one or more layers, draining the liquid from the solution to result in a rapidly drying solid material. This solid is collected and molded into a desired form prior to drying.

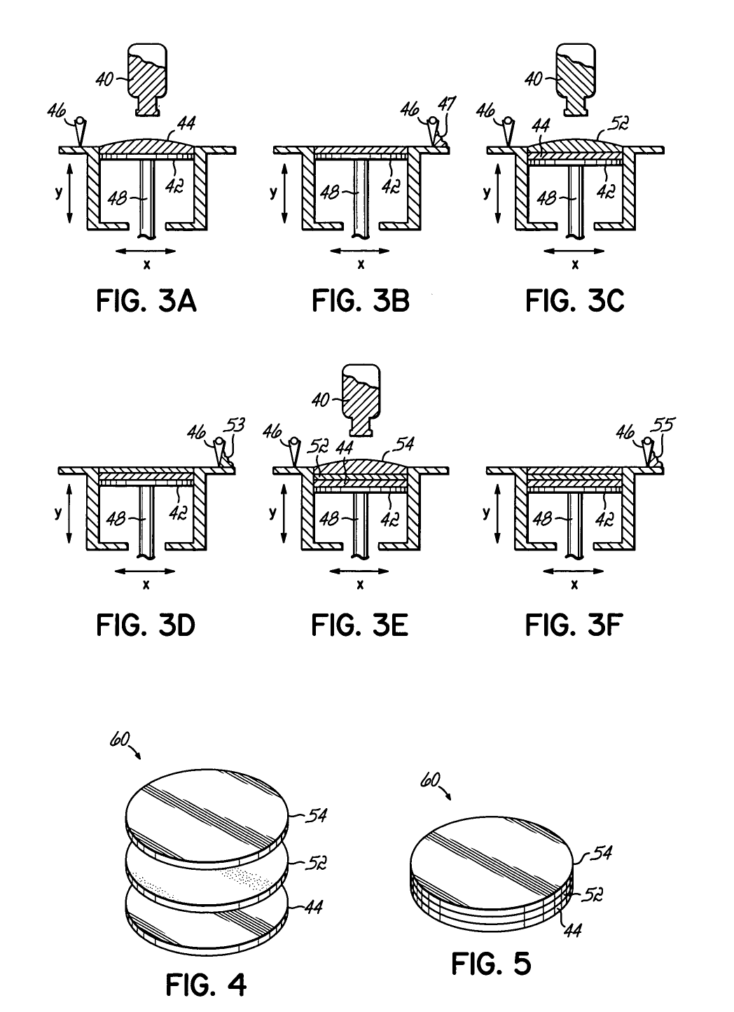

In the case that the RC and the medicament are incompatible in the solution form, the layup process is performed sequentially with independent, preformed RC gels, semi-dried or dried collagen matrices and medicament solutions, to form alternating layers of the RC and the medicament. With reference to FIGS. 3A-3F, there is shown an exemplary method of making a composition 30 of the present invention. As shown, a thick viscous solution of reconstituted collagen (RC) 40 is first poured onto a casting surface 42 of suitable collection equipment and compression or squeeze member 46 is pushed or otherwise moved across the RC 10 squeezing or compressing RC 10 into a thin layer 44 while forcing out most of the liquid portion of RC10 through drain 48. The surface 42 may be sufficiently designed and configured for draining liquid to drain 48. As illustrated in FIG. 3B, a thin layer 44 may require that member 46 carry away excess RC 47. Next, an amount of a medicament composition or solution 50 (e.g. API) is poured onto layer 44 to form a second layer. The casting surface 42 is lowered vertically with layer 44 to provide a volume to capture the medicament 50 for forming the second layer (See FIG. 3C). Then, the second layer 52 is also compressed or squeezed with member 46 to force liquid from medicament solution 50 through drain 48 thereby forming second layer 52 of a medicament, upon the first RC layer 44. Again, as shown in FIG. 3D, additional material 53 might be carried away. Another layer 54 of RC is similarly formed to sandwich medicament layer 52 between layers 44 and 54, as shown in FIGS. 3E and 3F. FIG. 4 illustrates an exploded view of the composition 60 formed by the method shown in FIGS. 3A-3F. FIG. 5 illustrates the final product composition formed with the multiple layers together.

Another suitable method for preparing the fibrous composition in accordance with the present invention is a printing process. With reference to FIG. 6, there is shown an exemplary method of printing successive layers of RC followed by a medicament in a desired amount. As shown, a layer 70 of RC may be printed with a protein printing head 72 and after the RC layer has dried, a layer 74 of a medicament may be printed on the layer 70 of the RC. Thus, in this manner sequential layers 70 of RC, or specific areas of the RC layer(s) 70, can be printed over with the medicament material to form a sandwich type of composition 76, as illustrated in FIG. 7, comprising two RC layers sandwiching the medicament layer. This printing method allows one to uniformly distribute and/or control the amount of the medicament on RC layer 70.

The printing process disclosed in FIG. 6 may be used and selectively controlled to vary the distribution and/or amount of the medicament in the product 76. For example, a greater amount of medicament might be deposited in one location of layer 70, such as the center, then at another location, such as the periphery. Furthermore, different API's might be used in different areas of the product 6. For example, one API might be deposited in one section, quadrant or one half of the product, while another different API might be deposited on a different section, quadrant or half of the product. The final composition 76, as shown in FIG. 7, can further be perforated, scored, and/or marked, such as with lines 80, to indicate dosage, amounts and/or other desired information.

Yet another suitable method of forming the fibers or threads is with the process of extrusion, such as electrostatic extrusion. Extrusion may generally be accomplished without spinning and/or melting any of the extruded components. Two or more extruded streams of some or all of the components of the composition may be converged and mixed to form threads or fibers utilized in the composition. The extruded fibers or filaments may then be formed in a spool or processed in multiple other ways. For example, the fibers may then be aligned into sheets. The sheets may be stacked one on top of the other in layers. For example, each fiber in a sheet or layer may be oriented in one direction, i.e., horizontal or up and down, whereas fibers in another sheet may be oriented in a vertical nature, or generally perpendicular relative the orientation of fibers in the adjacent sheet(s). Stacking of layers may involve alternating horizontal and vertically oriented fiber sheets or successive sheets may either be all horizontally oriented fibers or all vertically oriented fibers. Other combinations such as to form woven and cross-woven layers of fibers or threads, as appreciated by those skilled in the art, are also contemplated herein.

By virtue of being fibrous, the composition may then be compressed into desirable unique shapes. For example, the sheets may be compressed into multi-layered “blankets”, and them molded into desirable shapes, including without limitation, a biconvex shape, a biconcave shape, a flattened shape, and the like. The particular orientation of layers will generally affect the tensile strength and/or the ability of the sheets or layered blanket to absorb liquid.

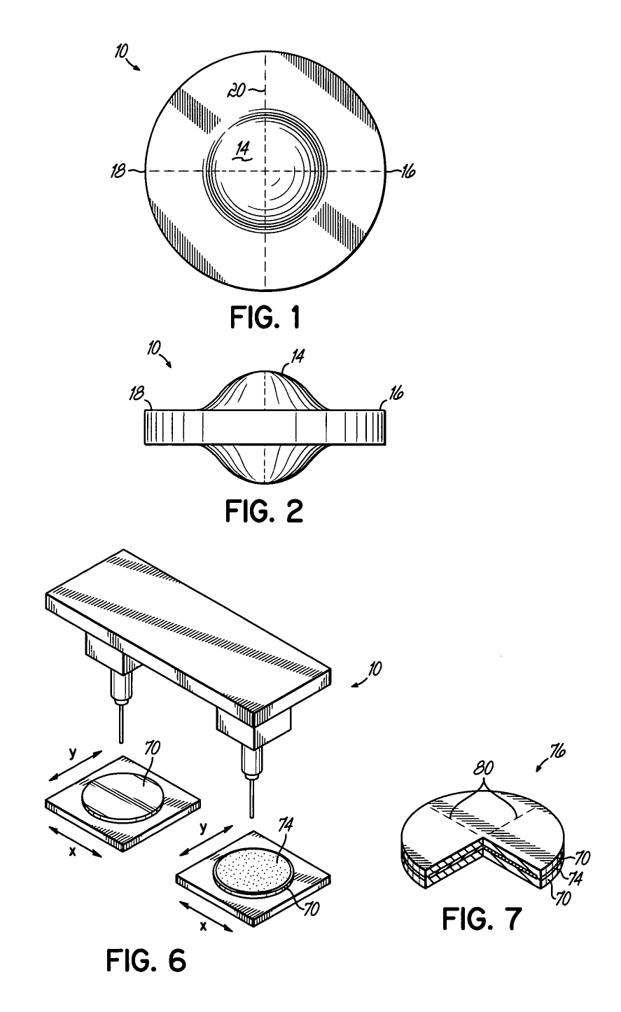

With reference to FIG. 1, there is shown a top-down view of an exemplary final shape of the fibrous composition. As shown, composition 10 is circular in shape. Composition 10 includes a central area 14 and terminal or peripheral areas 16,18. Central area 14 generally contains a concentration of collagen-based fibers and medicament in a desired weight ratio. Composition 10 may be further perforated and/or marked as desired. For example, score lines 21 divide composition 10 into quadrants.

With reference to FIG. 2, there is shown a side view of the exemplary form of the composition illustrated in FIG. 1. As shown, central area 14 is elevated or raised relative to terminal or peripheral areas 16,18. Terminal areas 16,18 are more compressed and flattened. In that sense, the composition takes on a fusiform shape.

The composition, once formed into desired shapes, could be further processed utilizing kiss-cut technology to perforate or separate desired amounts of the composition for dose titration and dose administrative purposes in accordance with another aspect of the invention. Indented scoring of the composition makes accurate tearing or cutting of the composition easier. Accordingly, an individual dose may be easily and conveniently obtained by simply cutting or severing scored sections of the composition. Alternatively, layers, sheets or blankets of the fibrous composition may be made commercially available and may be further processed to contain or “wrap” a pill or tablet.

The fibrous composition or matrix may be configured with an API that is released immediately upon ingestion of the composition. Wrapped therein may be a solid pill, tablet or other dosage form which is then ingested to release its API in a more delayed fashion in the gut. The API's between the fibrous composition and the other dosage form (e.g. pill/tablet) might be the same or different. Furthermore, multiple API's might be incorporated into each dosage form.

Each sheet and/or blanket of fibers may be “marked” to indicate the nature and/or amount of the medicament, the dosage schedule, day indicia, time indicia, and the like, as desired. The markings may also indicate the content of each scored section of the composition. The ability to mark the composition provides the advantage of allowing the manufacturer to be in compliance with FDA regulations and other identification requirements. The composition can be marked with FD&C approved food coloring or other GRAS items into halves, quarters, or other dose sizes. The quarter markings are generally suitable for solid oral medications.

The present invention will be further appreciated in light of the following example.

The following is an example of a fibrous, collagen-based, medicament-containing composition of the present invention prepared by a gel-casting method. Collagen in an acidic aqueous solution at 3 mg/ml is dialyzed in the cold against a 32.7 mM phosphate buffer solution at a pH range from about 7.0 to 7.4. This solution is cast into molds and the temperature increased to about 20° C.-35° C. After a period of time, typically about several minutes, the solution changes in turbidity as the collagen begins to undergo molecular self-assembly. This process is called fibrillogenesis, or reconstitution, and is completed when all the free collagen molecules have become aggregated to one another. The resulting aggregate is a high water content, gel matrix containing consolidated collagen fibrils, produced by a process generally referred to as thermal gelation. The fibril structure is normally formed in a random non-woven pattern, but may be influenced by convection, shear flow or electromagnetic fields to generate oriented structures having unique properties, as disclosed in U.S. Pat. No. 4,544,516, which disclosure is incorporated herein by reference in its entirety.

Selected medicaments, such as an API, may be added to the collagen solution prior to gelation, or they may be dialyzed or otherwise infiltrated into the formed aqueous gels by known methods, prior to drying the gel. The cast gels are then generally dried by freeze-drying or air-drying. Freeze drying provides a high surface area, porous collagen-based matrix suitable for the oral medicament delivery system of the present invention. Portions of a cast freeze-dried film or individually molded sections may be pressed, laminated or otherwise mechanically modified to form desired shapes for administration to a patient.

The medicament may be added before or after the gel is shaped to provide the final form of the composition administered in the oral medicament delivery system of the invention. Shaping or lamination may be facilitated by rewetting the dried matrix or by adding liquid collagen to the matrix and re-drying while molding the matrix to the desired shape.

By virtue of the foregoing, the present invention provides a medication delivery system for orally administering a medicament to a patient. The invention takes advantage of the desirable properties of collagen protein, which have been previously utilized in a variety of useful medical applications, while addressing weaknesses of traditional oral delivery methods. More specifically, the invention provides a composition constructed from a matrix of collagen or collagen-based fibers carrying one or more medicaments, and assembled in such a way as to disperse or dissolve on presentation to the buccal mucous membrane of the oral cavity. As such, the invention overcomes difficulties with traditional methods of oral administration, such as those involving swallowing of a pill, tablet, capsule, or other solid dosage form. The fibrous, collagen-based composition is flexible, readily soluble in the oral environment, and may be additionally appealing with sweeteners, shape, colors, taste enhancers, taste maskers, and the like, to render it more appealing for ingestion to both children and adults. Dose titration of the composition is possible by its physical structure and characteristics. Specifically, doses may be titrated by administering segmented portions of the fibrous matrix composition by simply cutting them with a pair of scissors or a knife.

Further, addition of a low dose liquid medication onto and into the composition may create a single unit swallowed medication. Particularly, forms of the composition having larger centers that are less compacted generally contain more space or air between fibers or individual filaments thereby allowing for absorption of the liquid. In that sense, the composition acts as a sponge to absorb and hold the liquid medication. The liquid might include the same API as an API incorporated into the fibrous matrix. Alternatively, it may be different. Still further, the liquid medication might be the only API in the overall product.

Another advantage is that the composition, by virtue of its flexible fibrous matrices, has the ability to “wrap around” another dry medication, such as a pill or powder, to create a single unit swallowed medication. Molding of the composition into desirable forms, as well as having self-adhering properties by virtue of other desired excipients therein along the edges of the composition, also allows for the composition of a single unit swallowed medication. Thus, the present invention enhances ingestion of a medicament dose and, therefore, improves patient compliance with ingestion in accordance with a medication regimen.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is, therefore, not limited to the specific details, representative apparatus, method, and examples described. Accordingly, departures may be made from such details without departing from the scope or spirit of Applicant's general inventive concept.

1. An oral medicament delivery composition comprising a flexible matrix, said matrix formed of a plurality of fibers comprising a collagen-based carrier and a medicament, the composition orally dissolvable to deliver a unit dose of the medicament.

2. The composition of claim 1 wherein the collagen-based carrier comprises chemically modified collagen.

3. The composition of claim 1 wherein the carrier to medicament weight ratio in each fiber is in the range from about 1:50 to about 50:1.

4. The composition of claim 1 wherein the carrier to medicament weight ratio in each fiber is in the range from about 1:1.

5. The composition of claim 1 having multiple layers of fibers.

6. The composition of claim 1 compressed into one of a biconvex shape, a biconcave shape, a flattened shape and a fusiform shape.

7. The composition of claim 1 including individual sections, each section comprising a unit dose of the medicament.

8. The composition of claim 1 wherein the medicament is an agent chosen from an opioid analgesic agent, a non-opioid analgesic agent, an anti-inflammatory agent, an antitussive agent, an antipyretic agent, an antibiotic agent, an antimicrobial agent, a steroidal agent, an amphetamine stimulant agent, a non-amphetamine stimulant agent, a laxative agent, an anorexic agent, an antihistaminic agent, an antiasthmatic agent, an antidiuretic agent, an antiflatulant agent, an antimigraine agent, an antispasmodic agent, an antidiabetic agent, a respiratory agent, a sympathomimetic agent, an H₂ blocking agent, an antihyperlipidemic agent, an antihypercholesterol agent, a cardiotonic agent, a vasodilating agent, a vasoconstricting agent, a sedative agent, a hypnotic agent, an anticonvulsant agent, a muscle relaxing agent, an antipyschotic agent, an antianxiolytic agent, an antihyperactive agent, an antihypertensive agent, an antitumor agent, a soporific agent, a tranquilizer, a decongestant, a beta-blocker, a non-steroidal hormone, an herbal agent, an enzyme, a humoral agent, a madriatic agent, a psychic energizer, a vitamin, a mineral, a dietary supplement, and combinations thereof.

9. The composition of claim 1 wherein the medicament is present in an amount ranging from about 1 mg to about 100 mg.

10. The composition of claim 1 further comprising an excipient selected from the group consisting of a sugar, a binding agent, non-effervescent disintegrants, a coloring agent, a flavoring agent, a taste enhancing agent, a taste masking agent, an oral dispersing agent, a stabilizer, a preservative, a diluent, a filler, a compaction agent, an effervescent disintegration agent and combinations thereof.

11. The composition of claim 1 further comprising a muco-adhesive in an amount sufficient to adhere the composition to the buccal membrane of a patient.

12. The composition of claim 1 wherein the medicament is incorporated into each fiber in the matrix.

13. The composition of claim 1 wherein the medicament is distributed on the flexible matrix.

14. The composition of claim 1 comprising layers of the medicament sandwiched between layers of the flexible matrix.

15. The composition of claim 1 further comprising a second dosage formulation independent of the flexible matrix.

16. An oral medicament delivery system comprising the composition of claim 1.

17. An oral medicament delivery system comprising a pharmaceutical composition including
at least one layer formed of a plurality of collagen-based fibers; and
a medicament,
the composition orally dissolvable to deliver a unit dose of the medicament to the oral cavity of a patient.

18. The delivery system of claim 16 wherein the composition includes a layer of the medicament between two layers of collagen-based fibers.

19. The composition of claim 16 wherein the weight ratio of fiber to medicament is in the range from about 1:50 to about 50:1.

20. The composition of claim 16 wherein the weight ratio of fiber to medicament is in the range from about 1:1.

21. The composition of claim 16 wherein the medicament is an agent chosen from an opioid analgesic agent, a non-opioid analgesic agent, an anti-inflammatory agent, an antitussive agent, an antipyretic agent, an antibiotic agent, an antimicrobial agent, a steroidal agent, an amphetamine stimulant agent, a non-amphetamine stimulant agent, a laxative agent, an anorexic agent, an antihistaminic agent, an antiasthmatic agent, an antidiuretic agent, an antiflatulant agent, an antimigraine agent, an antispasmodic agent, an antidiabetic agent, a respiratory agent, a sympathomimetic agent, an H₂ blocking agent, an antihyperlipidemic agent, an antihypercholesterol agent, a cardiotonic agent, a vasodilating agent, a vasoconstricting agent, a sedative agent, a hypnotic agent, an anticonvulsant agent, a muscle relaxing agent, an antipyschotic agent, an antianxiolytic agent, an antihyperactive agent, an antihypertensive agent, an antitumor agent, a soporific agent, a tranquilizer, a decongestant, a beta-blocker, a non-steroidal hormone, an herbal agent, an enzyme, a humoral agent, a madriatic agent, a psychic energizer, a vitamin, a mineral, a dietary supplement, and combinations thereof.

22. The composition of claim 16 wherein the fibers comprise chemically modified collagen.

23. A method of orally delivering a medicament to a patient, the method comprising:
administering a pharmaceutical composition to the oral cavity of a patient, the composition comprising a flexible matrix formed of a plurality of fibers comprising a collagen-based carrier and a medicament, and the composition orally dissolvable to deliver a unit dose of the medicament to the patient.

24. The method of claim 23 further comprising combining the composition with an aqueous-based liquid prior to administering the composition to the patient.

25. The method of claim 23 further comprising administering a second pharmaceutical formulation in combination with the composition.

26. The method of claim 25 wherein administering the second pharmaceutical formulation in combination comprises administering the second pharmaceutical formulation simultaneously or sequentially with the composition.

27. The method of claim 23 wherein the patient is one of a child and an adult.

28. A method of formulating an orally dissolvable medicament composition, the method comprising:
forming multiple fibers from a collagen-based material; and
combining a medicament with the fibers to formulate the orally dissolvable medicament composition.

29. The method of claim 28 further comprising providing reconstituted collagen for forming the multiple fibers.

30. The method of claim 28 further comprising compressing layers of multiple fibers with the medicament to formulate the orally dissolvable medicament composition.
